(19) United States
(12) Patent Application Publication
(10) Pub. No.: US 2025/0169842 A1
(43) Pub. Date: May 29, 2025

(54) BILE DUCT/PANCREATIC DUCT TREATMENT METHOD AND ENDOSCOPIC INSTRUMENT

(71) Applicant: OLYMPUS CORPORATION, Tokyo (JP)

(72) Inventors: Hirokazu YOKOTA, Hachioji-shi (JP); Shuichi ITO, Sagamihara-shi (JP); Ko TAKITA, Hachioji-shi (JP); Tomoyasu HASEGAWA, Hachioji-shi (JP)

(73) Assignee: OLYMPUS CORPORATION, Tokyo (JP)

(21) Appl. No.: 18/762,807

(22) Filed: Jul. 3, 2024

Related U.S. Application Data
(60) Provisional application No. 63/603,849, filed on Nov. 29, 2023.

(51) Int. Cl. A61B 17/32; A61B 1/05; A61B 1/07; A61B 1/32; A61B 17/00

(52) U.S. Cl. CPC A61B 17/320016; A61B 1/05; A61B 1/07; A61B 1/32; A61B 2017/00278; A61B 2017/00818

(57) ABSTRACT

A bile duct/pancreatic duct treatment method includes: positioning an endoscope having a channel from a duodenum to an opening of a papilla; inserting an imaging wire having an imaging unit at its distal end through the channel from the opening to a common duct; inserting the imaging wire into one of cavities of a bile duct and a pancreatic duct; observing the one of the cavities using the imaging unit; and if treatment with a treatment tool is required, inserting an access port through which the treatment tool can be inserted along the imaging wire into the one of the cavities.

### BACKGROUND

### Field of the Invention

The present invention relates to a bile duct/pancreatic duct treatment method and an endoscopic instrument.

### Description of Related Art

Procedures are performed to insert medical devices such as guidewires into the bile duct or pancreatic duct. Since the bile duct or pancreatic duct has narrowed or meandering parts, it is difficult to insert medical devices into the bile duct or pancreatic duct. As described in U.S. Pat. No. 4,979,496, etc., medical devices that are easy to insert into the bile duct or pancreatic duct have been devised.

### SUMMARY

A bile duct/pancreatic duct treatment method according to a first aspect of the present invention includes the steps of: positioning an endoscope having a channel from a duodenum to an opening of a papilla; inserting an imaging wire having an imaging unit at its distal end through the channel from the opening to a common duct; inserting the imaging wire into one of cavities of a bile duct and a pancreatic duct; observing the one of the cavities using the imaging unit; and if treatment with a treatment tool is required, inserting an access port through which the treatment tool can be inserted along the imaging wire into the one of the cavities.

An endoscopic instrument according to a second aspect of the present invention includes: an imaging wire having an imaging unit at its distal end; and an access port through which the imaging wire can be inserted, wherein the imaging wire and the access port are relatively movable along a longitudinal direction of the endoscopic instrument.

### BRIEF DESCRIPTION OF THE DRAWINGS

FIG. 1 is an overall view of the endoscopic treatment system according to a first embodiment.

FIG. 2 is an overall view showing an instrument of the endoscopic treatment system.

FIG. 3 is a perspective view of a distal part of the instrument.

FIG. 4 is a view showing a bile duct to be treated.

FIG. 5 is a view showing an imaging wire insertion step.

FIG. 6 is a view showing an image captured by the imaging unit of the instrument.

FIG. 7 is a view showing the imaging wire insertion step.

FIG. 8 is a view showing an access tube insertion step.

FIG. 9 is a view showing a treatment step.

FIG. 10 is a view showing an access tube placement step in a bile duct/pancreatic duct treatment method according to a second embodiment.

FIG. 11 is a view showing a first insertion step of the imaging wire.

FIG. 12 is a view showing a first insertion step of the access tube.

FIG. 13 is a view showing an observation step.

FIG. 14 is a view showing a second insertion step of the access tube.

FIG. 15 is a view showing a modified example of the access tube placement step.

FIG. 16 is a view showing a modified example of the access tube.

FIG. 17 is a diagram showing an endoscopic instrument relating to a third embodiment.

FIG. 18 is a diagram showing a treatment tool insertion step in a bile duct/pancreatic duct treatment method relating to the third embodiment.

FIG. 19 is a diagram showing a treatment step.

### DETAILED DESCRIPTION

### First Embodiment

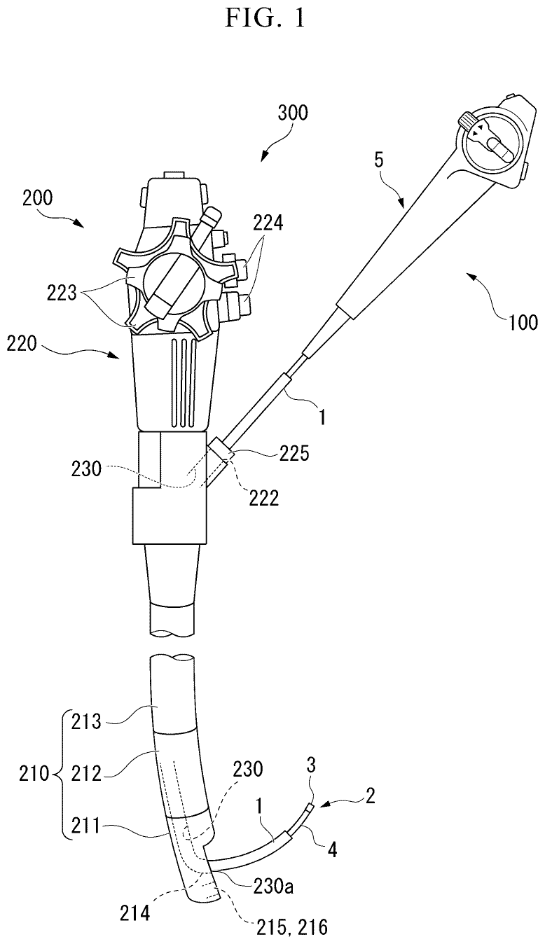

A bile duct and pancreatic duct treatment method according to a first embodiment of the present invention will be described with reference to FIGS. 1 to 9.

### [Endoscope Treatment System 300]

FIG. 1 is an overall view of an endoscopic treatment system 300. As shown in

FIG. 1, the endoscopic treatment system 300 includes an endoscopic instrument 100 and an endoscope 200. The endoscopic instrument 100 is inserted into the endoscope 200 for use.

### [Endoscope 200]

The endoscope 200 is a known side-viewing flexible endoscope and includes a long insertion section 210 and an operating section 220 provided at the proximal end of the insertion section 210. The endoscope 200 may also be a front-viewing flexible endoscope.

The insertion section 210 has a distal end rigid section 211 provided at the distal end, a bending section 212 provided at the proximal end of the distal end rigid section 211 that can be bent, and a flexible tube section 213 provided at the proximal end of the bending section 212. An imaging unit 216 having a light guide 215 and an image sensor is provided on the side of the distal end rigid section 211 in a state where it is exposed to the outside.

The insertion section 210 is formed with a channel 230 for inserting the endoscopic instrument 100. The distal end 230a of the channel 230 opens on the side of the distal end rigid section 211. The proximal end of the channel 230 extends to the operation section 220.

The distal end rigid section 211 of the channel 230 is provided with a lifting stand 214. The proximal end of the lifting stand 214 is rotatably supported by the distal end rigid section 211. The elevator control wire (not shown) is fixed to the distal end of the elevator 214 and extends to a proximal end side through the insertion section 210.

The bending section 212 is configured to bend freely in the vertical and horizontal directions. The distal end of the control wire is fixed to the distal side of the bending section 212. The control wire extends to the operation section 220 through the insertion section 210.

The proximal end side of the operation section 220 is provided with a knob 223 for controlling the control wire and a switch 224 for operating the imaging unit 216, etc. The surgeon can bend the bending section 212 in the desired direction by operating the knob 223.

The distal side of the operation section 220 is provided with a forceps port 222 that communicates with the channel 230. The surgeon can insert the endoscopic instrument 100 from the forceps port 222. A forceps plug 225 is attached to the forceps port 222 to prevent leakage of bodily fluids.

### [Endoscopic Instrument 100]

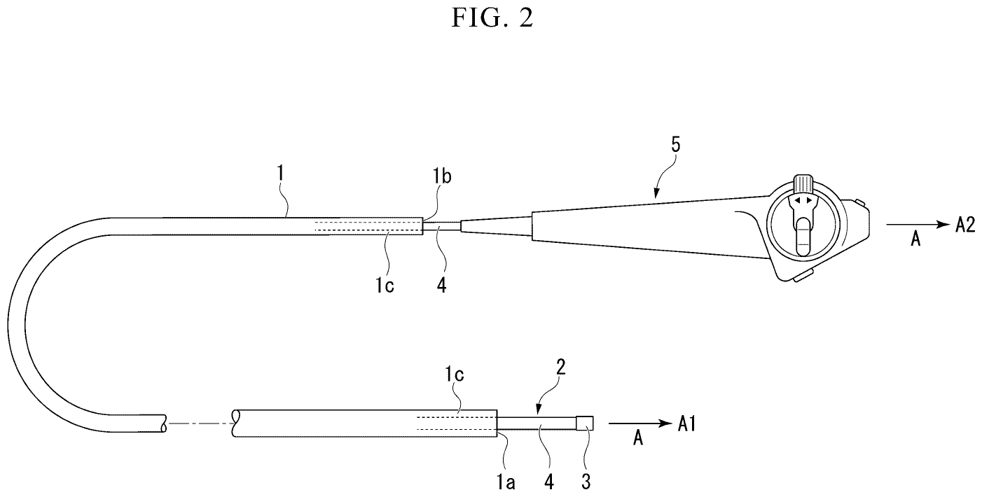

FIG. 2 is an overall view of the endoscopic instrument 100.

The endoscopic instrument 100 (also called the instrument 100) includes an access tube 1 and an imaging wire 2. In the following description, in the longitudinal direction A of the instrument 100, the side that is inserted into the patient's body is called the “distal side A1”, and the opposite side to the distal side A1 is called the “proximal side A2”.

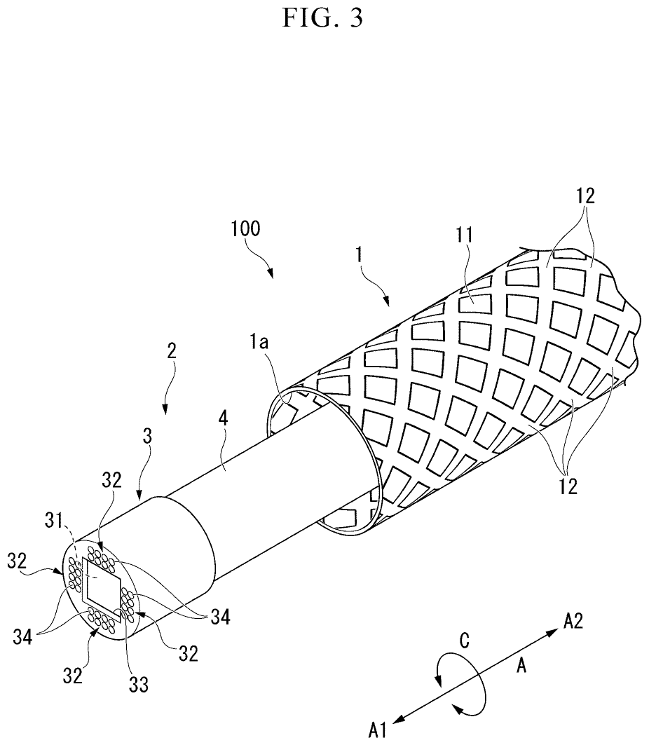

FIG. 3 is a perspective view of the distal end of the instrument 100.

The access tube (access port) 1 is a long flexible cylindrical member that extends from a distal opening la to a proximal opening 1b. The access tube 1 can be inserted into the channel 230 of the endoscope 200. A lumen 1s through which the imaging wire 2 can be inserted is formed inside the access tube 1. The inner diameter of the access tube 1 is, for example, 2 mm to 3 mm. The lumen 1s of the access tube 1 can be inserted with various treatment tools such as a high-frequency knife or a cannula. The access tube 1 can guide the imaging wire 2 and other treatment tools that pass through the lumen 1s.

The access tube 1 is a blade tube in which a blade 12 is disposed on a tube 11 made of resin. The blade 12 is made of a highly rigid metal or resin and imparts high torque transmission characteristics and kink resistance to the tube 11. Therefore, the access tube 1 has high torque transmission characteristics and kink resistance characteristics while having a small diameter. The access tube 1 is not limited to a blade tube and may be, for example, a tube made of metal or resin.

The imaging wire 2 passes through the access tube 1 and can protrude and retract from the distal opening la of the access tube to the distal side A1. The imaging wire 2 includes an imaging unit 3, a sheath 4, and an operating section 5.

The imaging unit 3 is provided at the distal end of the imaging wire 2. The imaging unit 3 includes an imaging section 31 and an illumination section 32.

The imaging unit 31 has a lens and an image sensor. The imaging signal generated by the imaging unit 31 is acquired by an external imaging device via a connection cable. The external imaging device generates an image based on the imaging signal and outputs the image to a monitor.

The imaging unit 31 is provided so as to be able to image the distal side A1 from an imaging frame 33 provided at the distal end of the imaging unit 3. The imaging frame 33 is formed in a square shape when viewed from the distal side A1. The imaging frame 33 may also be formed in a circular shape.

The illumination section 32 irradiates the distal side A1 with light supplied from the light source device via an illumination fiber 34 inserted through the sheath 4. The light irradiated by the illumination section 32 is white light or infrared light, such as special light used for NBI (Narrow Band Imaging) or RDI (Red Dichromatic Imaging). The irradiated light is also used for therapeutic purposes, such as irradiating a lesion or irradiating a photosensitive drug administered to the living body to cause a chemical reaction.

As shown in FIG. 3, the illumination sections 32 are arranged on all four sides of the imaging frame 33. Each of the illumination sections 32 arranged on all four sides has eight illumination fibers 34. The illumination fibers are made of, for example, glass or resin.

The arrangement position and number of the illumination fibers 34 are not limited to this.

The sheath 4 is a flexible, long cylindrical member. The imaging unit 3 is provided at the distal end of the sheath 4. The operation unit 5 is provided at the proximal end of the sheath 4. A lumen is formed inside the sheath 4 through which the imaging cable and the illumination fiber 34 can be inserted.

The operation unit 5 is provided at the proximal end of the sheath 4. The surgeon can advance and retract the imaging unit 3 along the longitudinal direction A by advancing and retracting the operation unit along the longitudinal direction A. The surgeon can rotate the imaging unit 3 in the circumferential direction C by rotating the operation unit 5 in the circumferential direction C relative to the longitudinal direction A.

### [Operation of the Endoscopic Treatment System 300]

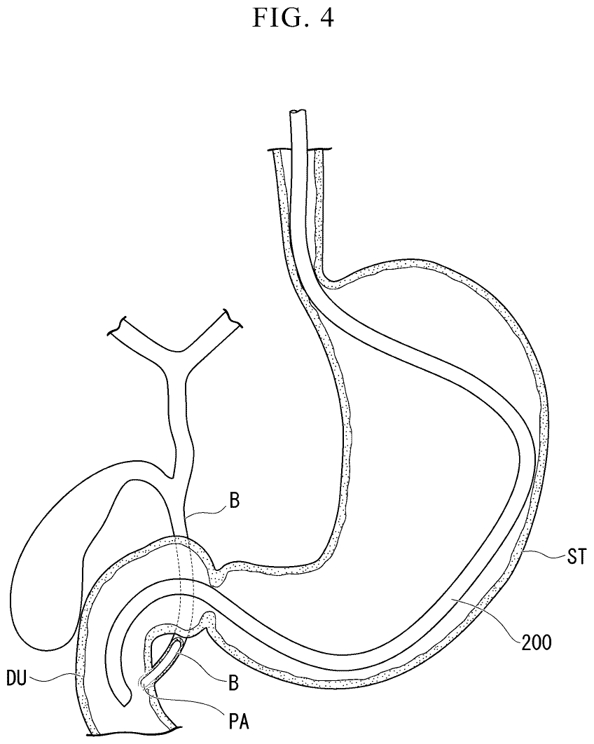

Next, a bile duct/pancreatic duct treatment method according to this embodiment will be described. Specifically, a procedure for observing and treating a diseased area D of the bile duct B will be described. FIG. 4 shows the bile duct B as a treatment target.

### <Step S1: Endoscope Insertion Step>

The surgeon inserts the insertion section 210 of the endoscope 200 into the patient's lumen from a natural opening such as the mouth. If necessary, the surgeon operates the operation unit 220 to bend the bending section 212. The surgeon inserts the distal end rigid section 211 of the endoscope 200 up to the duodenum DU. At that time, the surgeon positions the distal end rigid section 211 in a position that captures the opening PA1 of the papilla PA in front so that the protruding direction (or protruding axis) of the instrument 100 and the bile duct direction (or bile duct axis) are aligned. The surgeon may insert the insertion section 210 of the endoscope 200 into the patient's lumen using an overtube.

### <Step S2: Imaging Wire Insertion Step>

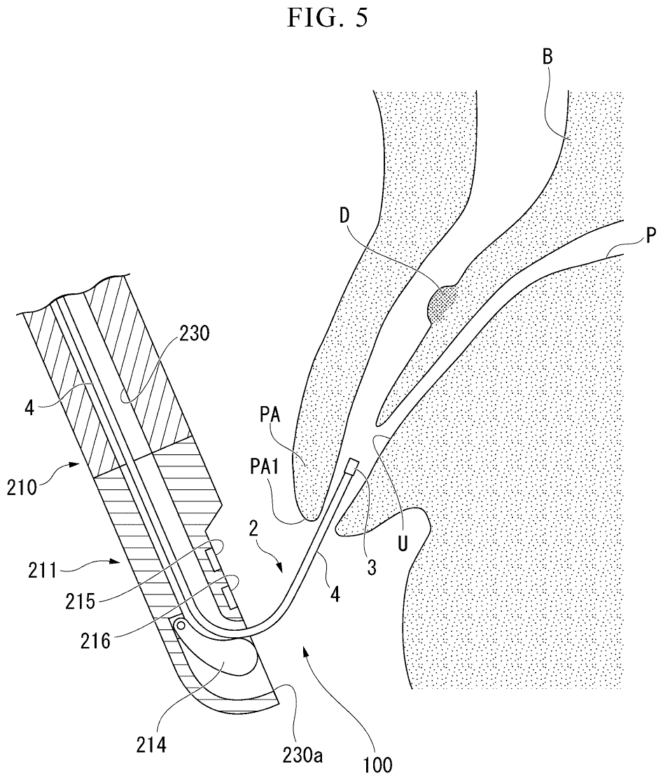

FIG. 5 shows an imaging wire insertion step.

The surgeon inserts the imaging wire 2 into the channel 230 of the endoscope 200. The surgeon protrudes the distal end of the imaging wire 2 from the distal end 230a of the channel 230, and operates the lifting base 214 to insert the imaging wire 2 from the opening PA1 of the papilla PA into the common duct U located in front of the bile duct B and the pancreatic duct P.

When inserting a conventional biliary-pancreatic endoscope into the opening PA1 of the papilla PA, the surgeon had to incise the opening PA1 of the papilla PA using a papilla incision knife of the cannula or the like. On the other hand, since the imaging wire 2 has a smaller outer diameter than the conventional biliary-pancreatic endoscope, the surgeon can insert the imaging wire 2 into the opening PA1 of the papilla PA without necessarily incising the opening PA1 of the papilla PA. Only in certain circumstances, such as when it is difficult to insert the imaging wire 2 into the opening PA1 of the papilla PA, the surgeon can incise the opening PA1 of the papilla PA using a cannula papilla incision knife or the like.

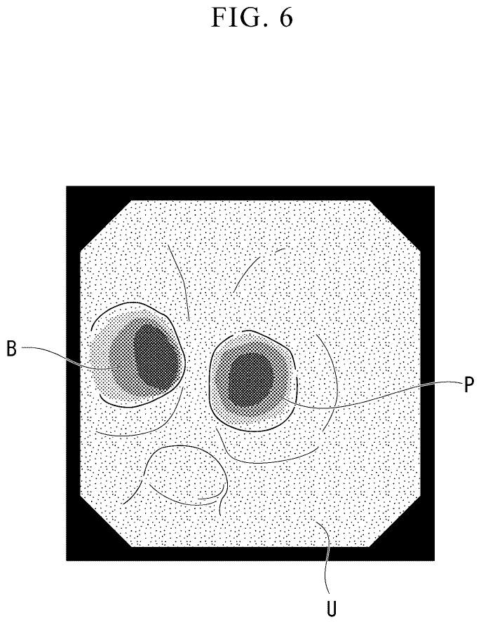

FIG. 6 shows an image captured by the imaging unit 3.

The surgeon inserts the distal end of the imaging wire 2 into the bile duct B of the treatment target, rather than the pancreatic duct P, by moving the distal end of the imaging wire 2 back and forth and rotating it while checking the image captured by the imaging unit 3. When the treatment target is a diseased part of the pancreatic duct P, the surgeon inserts the distal end of the imaging wire 2 into the pancreatic duct P of the treatment target, rather than the bile duct B.

When using a conventional biliary-pancreatic endoscope, the surgeon had to distinguish between the bile duct B and the pancreatic duct P while viewing an X-ray fluoroscopic image obtained by endoscopic retrograde cholangiopancreatography (ERCP), and) and insert the biliary-pancreatic endoscope into one of the cavities that is the treatment target. On the other hand, by inserting the imaging wire 2 into the common duct U, the surgeon can easily distinguish between the bile duct B and the pancreatic duct P while directly checking the image captured by the imaging unit 3, and insert the imaging wire 2 into one of the cavities to be treated.

The surgeon may inject contrast medium from the distal end opening la at the time of inserting the access tube 1 into the common duct U before the imaging wire insertion step. Alternatively, the surgeon may inject contrast medium from the opening PA1 of the papilla PA using a cannula before the imaging wire insertion step. In addition to the image captured by the imaging unit 3, the surgeon can confirm the shape of the bile duct/pancreatic duct and the distal end of the imaging wire 2 using X-ray contrast images obtained by endoscopic retrograde cholangiopancreatography (ERCP).

### <Step S3: Observation Step>

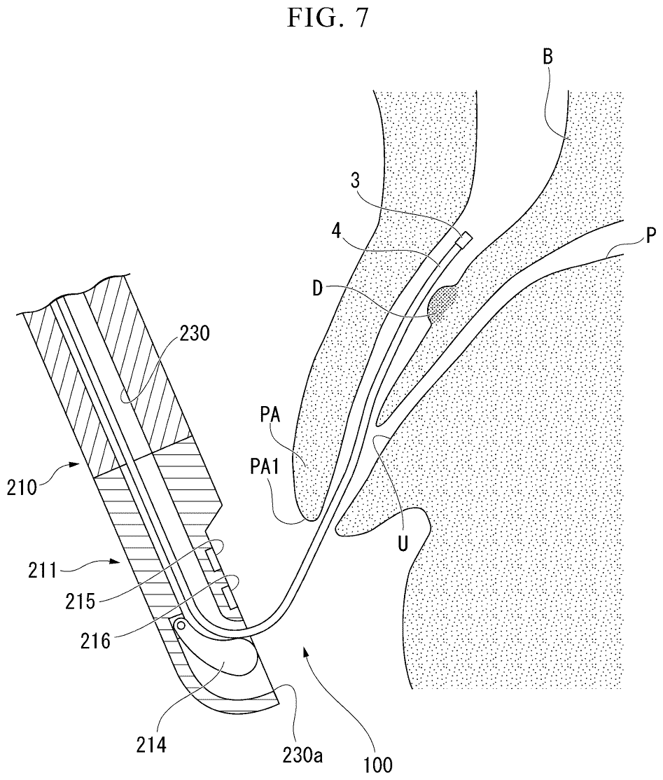

FIG. 7 is a diagram showing the imaging wire insertion step.

The surgeon observes the diseased area D of the bile duct B while advancing and retracting the imaging wire 2 inserted into the bile duct B. Specifically, the surgeon visually checks the images captured by the imaging unit 3 to see nodules and cysts present in the bile duct B. The surgeon also distinguishes the need for a biopsy diagnosis or treatment while checking the images captured by the imaging unit 3.

When observing the diseased area D using a conventional biliary-pancreatic endoscope, the surgeon has difficulty passing the biliary-pancreatic endoscope through the narrowed stenosis caused by the diseased area D. On the other hand, since the imaging wire 2 has a smaller outer diameter than the conventional biliary-pancreatic endoscope, the surgeon can easily pass the imaging wire 2 through the stenosis as shown in FIG. 7. Therefore, the surgeon can easily observe the bile duct B using the imaging wire 2.

### <Step S4: Access Tube Insertion Step>

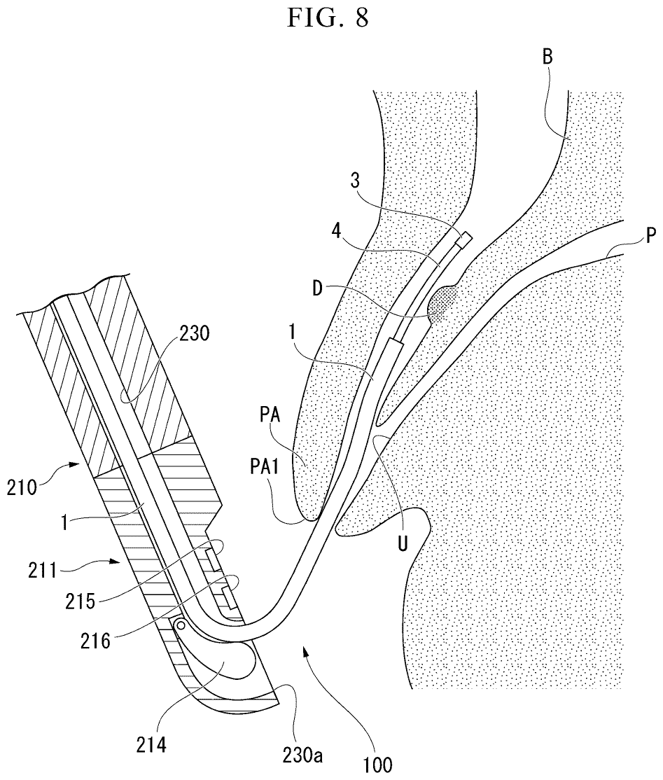

FIG. 8 is a diagram showing the access tube insertion step.

When the surgeon observes the diseased area D and determines that a biopsy diagnosis or treatment is necessary, the surgeon inserts the access tube 1 into the bile duct B along the imaging wire 2 that has already been inserted into the bile duct B.

Specifically, the surgeon inserts the imaging wire 2 into the lumen 1s of the access tube 1 and moves the access tube 1 to the distal side A1 relative to the imaging wire 2, thereby inserting the access tube 1 into the bile duct B.

### <Step S5: Treatment Step>

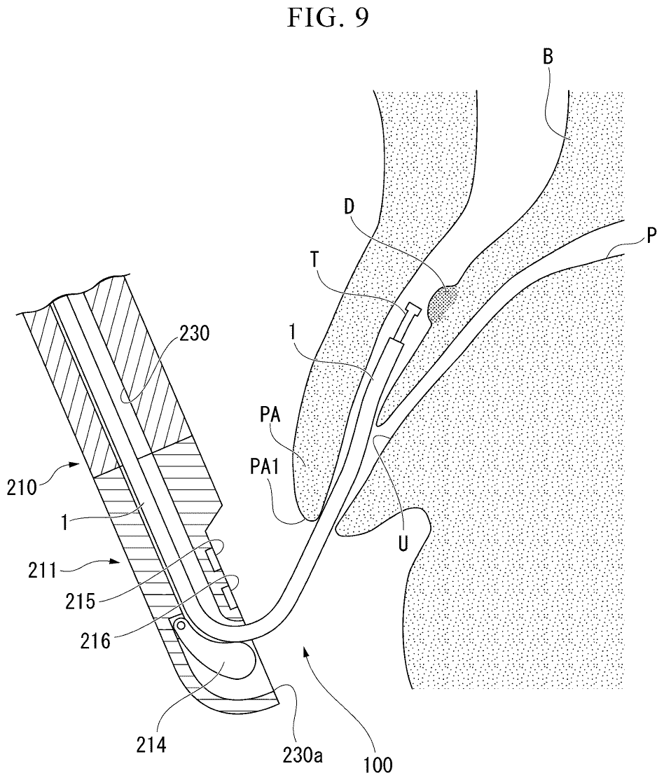

FIG. 9 shows the treatment step.

The surgeon removes the imaging wire 2 from the access tube 1 and inserts the treatment tool T (other treatment tools, guide wire, etc.) required for the treatment into the bile duct B along the access tube 1. Specifically, the surgeon inserts the treatment tool T into the lumen 1s of the access tube 1 and moves the treatment tool T to the distal side A1 relative to the access tube 1, thereby inserting the treatment tool T into the bile duct B. The surgeon uses the treatment tool T placed on the distal side A1 of the access tube 1 to treat the diseased area D.

If the treatment tool T can be inserted into the access tube 1 without removing the imaging wire 2 from the access tube 1, the surgeon does not need to remove the imaging wire 2 from the access tube 1.

After completing the treatment, the surgeon removes the treatment tool T, the access tube 1, and the endoscope 200.

According to the bile duct/pancreatic duct treatment method and endoscopic instrument 100 of this embodiment, the endoscopic instrument 100 and treatment tool T can be easily inserted into the bile duct B or pancreatic duct P. Since the access tube 1 and the imaging wire 2 are separate, only one of them can be inserted into the bile duct B or pancreatic duct P. The access tube 1 and the imaging wire 2 have smaller outer diameters than conventional bile duct pancreatic endoscopes in which an imaging mechanism and a treatment tool port are integrally provided, so that observation and treatment are less invasive. Furthermore, in conventional bile duct pancreatic endoscopes, a channel equivalent to the access tube 1 and an imaging unit are mounted on the bile duct pancreatic endoscope, so these could not be inserted independently. On the other hand, according to the bile duct/pancreatic duct treatment method and endoscopic instrument 100 of this embodiment, the access tube 1 and the imaging wire 2 are separate and can be inserted independently. As a result, only the imaging wire 2 with a small outer diameter is inserted into the bile duct, etc., and the diseased area D is observed, and then the access tube 1 for accessing the treatment tool T to the diseased area D can be inserted into the bile duct, etc., if necessary. Therefore, it is not necessary to insert the access tube 1 together with the imaging wire 2, and a less invasive procedure can be achieved. Furthermore, when observing the diseased area D, it is possible to insert only the imaging wire 2, which has a small outer diameter, into the bile duct, etc., making it possible to observe areas beyond the obliterated area and narrowed area of the bile duct and pancreatic duct that could not be reached with a conventional biliary-pancreatic endoscope.

The first embodiment of the present invention has been described above in detail with reference to the drawings, but the specific configuration is not limited to this embodiment, and design changes and the like that do not deviate from the gist of the present invention are also included. In addition, the components shown in the above-mentioned embodiment and modified examples can be appropriately combined to form a configuration.

### Modified Example 1-1

In the above embodiment, the distal end of the imaging unit 3 may have a bent tendency. The distal end of the imaging unit 3 that has a bent tendency can be easily controlled in the direction in which the distal end faces by rotating it, making it easy to insert it into the bile duct B or the pancreatic duct P.

### Second Embodiment

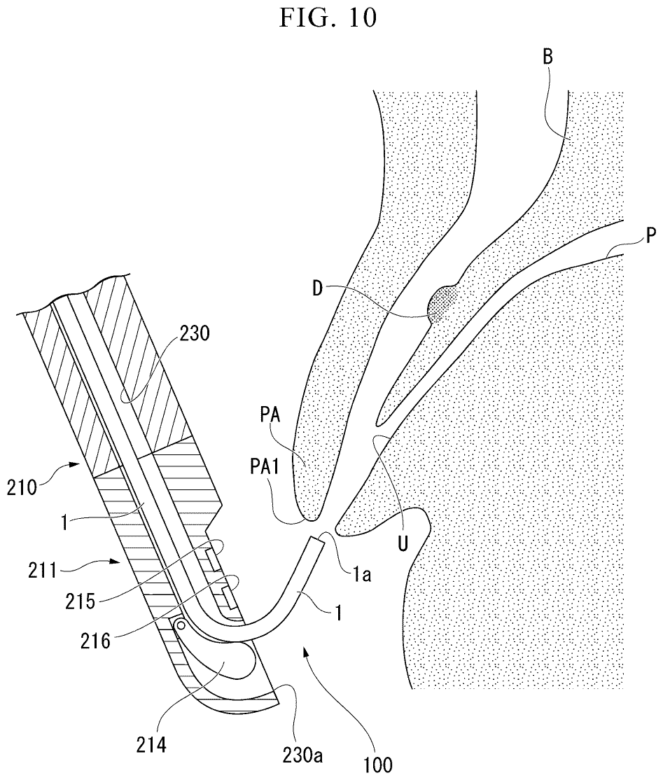

A method for treating the bile duct and pancreatic duct according to the second embodiment of the present invention will be described with reference to FIGS. 10 to 16. In the following description, components common to those already described are given the same reference numerals and redundant description will be omitted.

The bile duct/pancreatic duct treatment method according to this embodiment uses an endoscopic treatment system 300 including the endoscope 200 and the instrument 100 of the first embodiment. The bile duct/pancreatic duct treatment method according to this embodiment will be described. Specifically, the procedure for observing and treating the diseased part D of the bile duct B will be described.

### <Step S2-1: Endoscope Insertion Step>

The surgeon inserts the distal end rigid section 211 of the endoscope 200 into the duodenum DU, as in step S1 of the first embodiment.

### <Step S2-2: Access Tube Placement Step>

FIG. 10 is a diagram showing the access tube placement step.

The surgeon inserts the access tube 1 into the channel 230 of the endoscope 200. The surgeon protrudes the distal end of the imaging wire 2 from the distal end 230a of the channel 230, and positions the access tube 1 so that the distal end opening la of the access tube 1 faces the opening PA1 of the papilla PA by operating the lifting base 214.

### <Step S2-3: First Insertion Step of Imaging Wire>

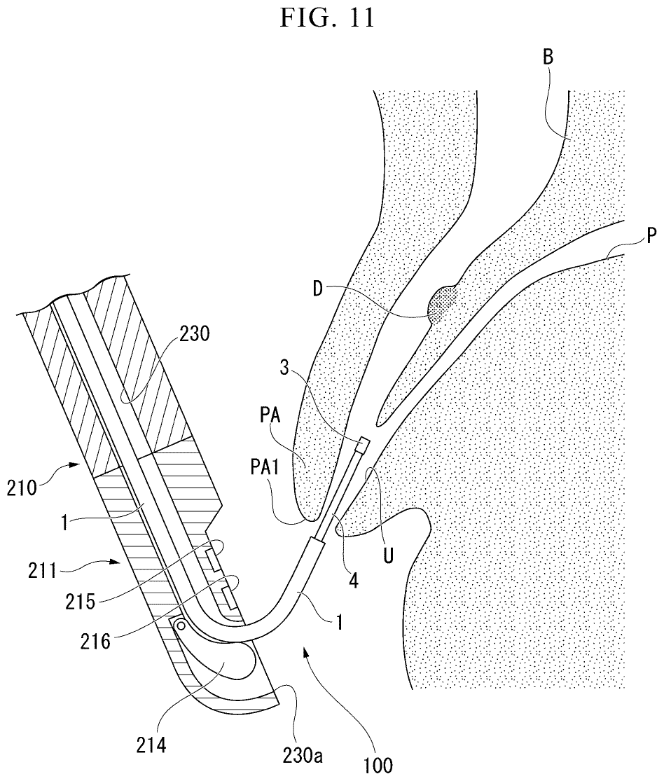

FIG. 11 is a diagram showing a first insertion step of the imaging wire.

The surgeon inserts the imaging wire 2 into the lumen 1s of the access tube 1. The surgeon protrudes the distal end of the imaging wire 2 from the distal end opening la of the access tube 1 by moving the imaging wire 2 to the distal side A1 relative to the access tube 1. The surgeon inserts the protruded distal end of the imaging wire 2 into the common tube U.

As in the first embodiment, the imaging wire 2 has a smaller outer diameter than a conventional biliary-pancreatic endoscope, so the surgeon can insert the imaging wire 2 into the opening PA1 of the papilla PA without necessarily incising the opening PA1 of the papilla PA. Only in certain circumstances, such as when it is difficult to insert the imaging wire 2 into the opening PA1 of the papilla PA, can the surgeon incise the opening PA1 of the papilla PA using a cannula papilla incision knife or the like.

### <Step S2-4: First Insertion Step of Access Tube>

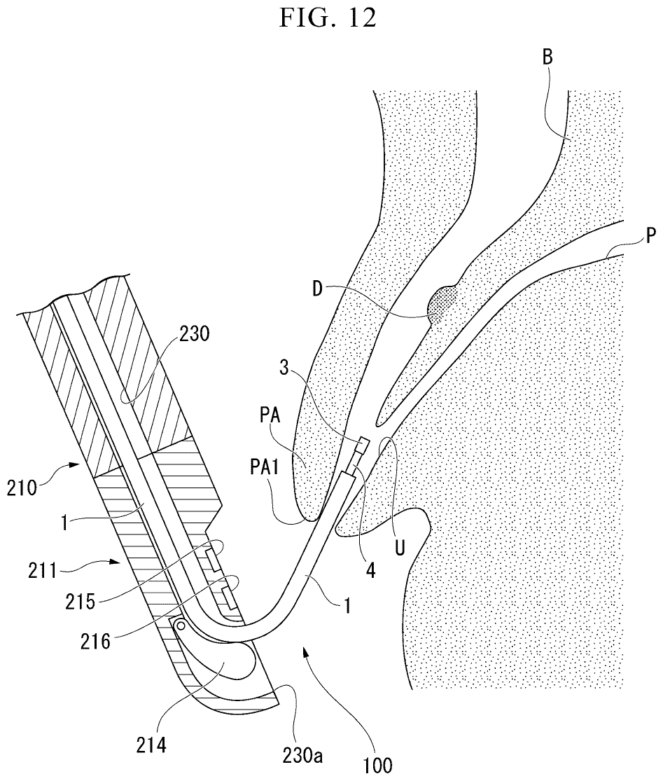

FIG. 12 shows a first insertion step of the access tube.

The surgeon inserts the access tube 1 into the bile duct B along the imaging wire 2 that has already been inserted into the common tube U. Specifically, the surgeon inserts the access tube 1 into the common tube U by moving the access tube 1 to the distal side A1 relative to the imaging wire 2 that passes through the lumen 1s of the access tube 1.

### <Step S2-5: Second Insertion Step of Imaging Wire>

While checking the image captured by the imaging unit 3, the surgeon advances and retreats the distal end of the imaging wire 2 and rotates it to insert the distal end of the imaging wire 2 into the bile duct B to be treated, not into the pancreatic duct P. When the treatment target is a diseased area of the pancreatic duct P, the surgeon inserts the distal end of the imaging wire 2 into the pancreatic duct P of the treatment target, not the bile duct B.

As in the first embodiment, the surgeon can easily distinguish between the bile duct B and the pancreatic duct P by inserting the imaging wire 2 into the common duct U while directly checking the image captured by the imaging unit 3 and insert the imaging wire 2 into one of the cavities that is the treatment target.

The surgeon may inject contrast medium from the distal end opening la at the time of inserting the access tube 1 into the common duct U. Alternatively, the surgeon may inject contrast medium from the opening PA1 of the papilla PA using a cannula before the access tube placement step. In addition to the image captured by the imaging unit 3, the surgeon can confirm the shape of the bile duct/pancreatic duct and the distal end of the imaging wire 2 using X-ray contrast images obtained by endoscopic retrograde cholangiopancreatography (ERCP).

### <Step S2-6: Observation Step>

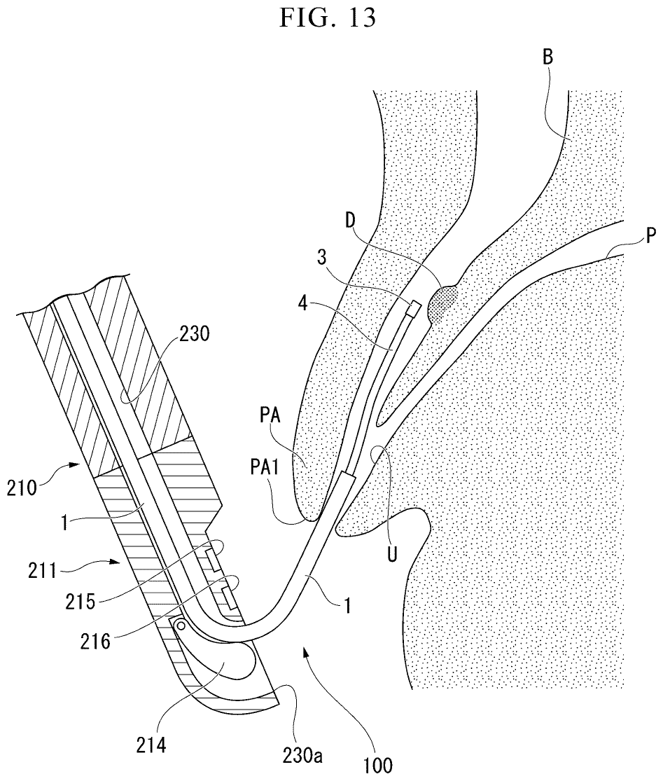

FIG. 13 is a diagram showing the observation step.

The surgeon observes the diseased area D of the bile duct B while advancing and retracting the imaging wire 2 inserted into the bile duct B, as in step S3 of the first embodiment.

Similar to the first embodiment, the imaging wire 2 has a smaller outer diameter than a conventional biliary-pancreatic endoscope, so the surgeon can easily pass the imaging wire 2 through the stenosis as shown in FIG. 13. This makes it easier for the surgeon to observe the bile duct B using the imaging wire 2.

### <Step S2-7: Second Insertion Step of Access Tube>

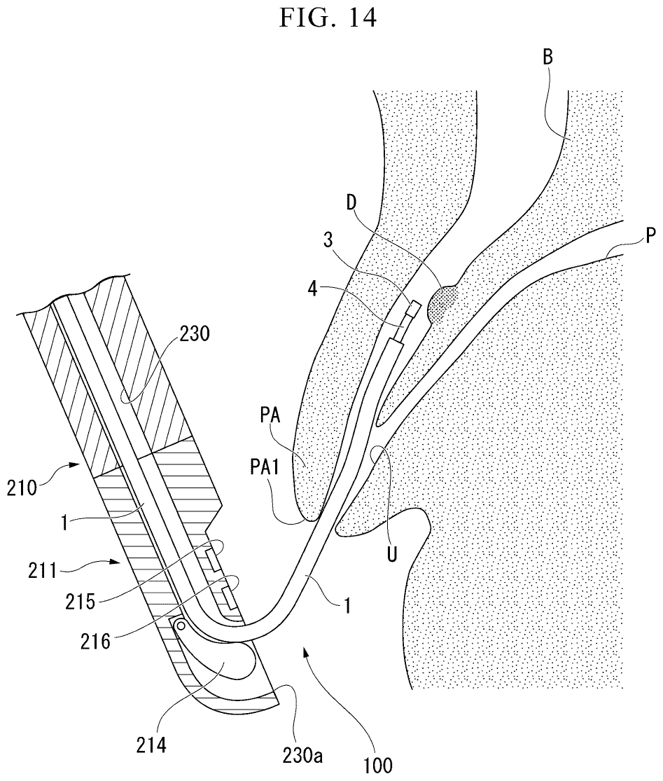

FIG. 14 is a diagram showing a second insertion step of the access tube.

If the surgeon observes the diseased area D and determines that a biopsy diagnosis or treatment is necessary, the surgeon inserts the access tube 1 into the bile duct B along the imaging wire 2 that has already been inserted into the bile duct B. Specifically, the surgeon inserts the access tube 1 into the bile duct B by moving the access tube 1 to the distal side A1 relative to the imaging wire 2 that passes through the lumen 1s of the access tube 1.

### <Step S2-7: Treatment Step>

The surgeon uses the treatment tool T to treat the diseased area D, as in step S5 of the first embodiment.

After completing the treatment, the surgeon removes the treatment tool T, the access tube 1, and the endoscope 200.

According to the bile duct/pancreatic duct treatment method and the endoscopic instrument 100 of this embodiment, the endoscopic instrument 100 and the treatment tool T can be easily inserted into the bile duct B or the pancreatic duct P. Since the access tube 1 and the imaging wire 2 are separate, only one of them can be inserted into the bile duct B or the pancreatic duct P. The access tube 1 and the imaging wire 2 have smaller outer diameters than conventional bile duct/pancreatic endoscopes in which an imaging mechanism and a treatment tool port are integrally provided, so that observation and treatment are less invasive. In addition, in the conventional biliary-pancreatic endoscope, the channel corresponding to the access tube 1 and the imaging unit are mounted on the biliary-pancreatic endoscope, and therefore these could not be inserted independently. On the other hand, in the biliary-pancreatic duct treatment method and endoscopic instrument 100 according to the present embodiment, the access tube 1 and the imaging wire 2 are separate and can be inserted independently. As a result, only the imaging wire 2 having a small outer diameter is inserted into the bile duct, etc., and the diseased area D is observed, and then, if necessary, the access tube 1 for accessing the treatment tool T to the diseased area D is inserted into the bile duct, etc. Therefore, it is not necessary to insert the access tube 1 together with the imaging wire 2, and a less invasive procedure can be realized. Furthermore, when observing the diseased area D, since only the imaging wire 2 having a small outer diameter can be inserted into the bile duct, etc., it is possible to observe the obliterated area of the biliary-pancreatic duct and the area beyond the narrowed area, which could not be reached by the conventional biliary-pancreatic endoscope.

The second embodiment of the present invention has been described above in detail with reference to the drawings, but the specific configuration is not limited to this embodiment, and design changes within the scope of the present invention are also included. In addition, the components shown in the above-mentioned embodiment and modified examples can be appropriately combined to form a configuration.

### Modified Example 2-1

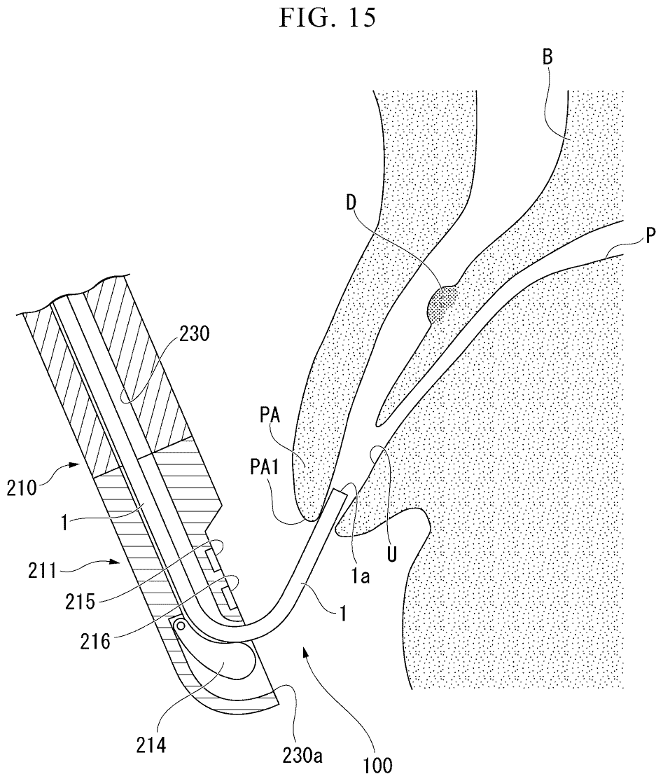

FIG. 15 is a diagram showing a modified example of the access tube placement step.

If it is difficult to insert the imaging wire 2 into the opening PA1 of the nipple PA in the first insertion step of the imaging wire of the above-mentioned embodiment, the surgeon may incise the opening PA1 of the nipple PA using a cannula nipple incision knife or the like. In this case, the surgeon may insert the distal end of the access tube 1 into the incised opening PA1 of the nipple PA in the access tube placement step, as shown in FIG. 15. This ensures that the insertion path of the imaging wire 2 into the common tube U can be secured.

### Modified Example 2-2

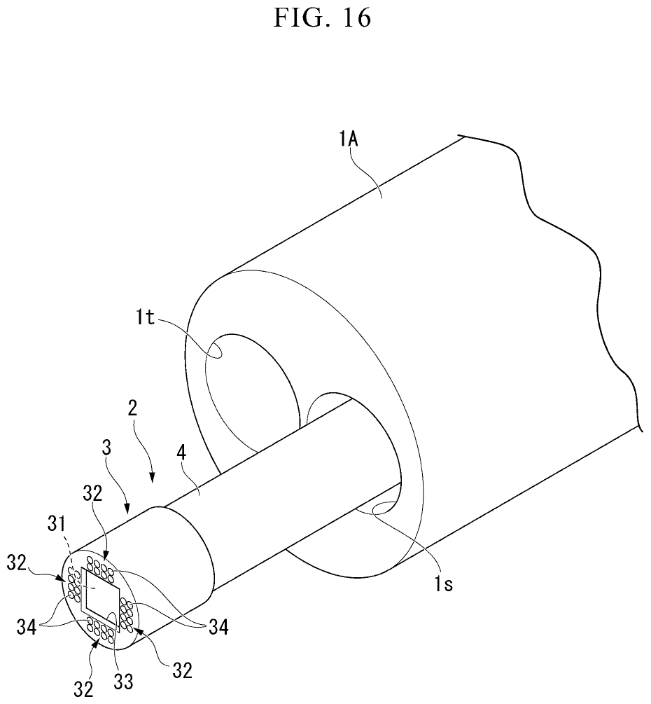

In the above-mentioned embodiment, the access tube 1 is a single-lumen tube. However, the shape of the access tube 1 is not limited. FIG. 16 is a diagram showing an access tube 1A, which is a modified example of the access tube 1. The access tube 1A is a multi-lumen tube and has a second lumen 1t in addition to the lumen 1s. The surgeon can insert the instrument 100 into the lumen 1s and the treatment tool T into the second lumen 1t, thereby allowing the surgeon to operate the instrument 100 and the treatment tool T without interference between them.

### Modification 2-3

The distal end of the access tube 1 may have a bending mechanism that can be actively bent. The bending mechanism of the access tube 1 can be operated from an operating unit provided on the proximal end side. In the second insertion step of the imaging wire, the surgeon bends the bending mechanism by operating the operating unit so that the distal end of the access tube 1 faces one of the cavities (bile duct B or pancreatic duct P) that is the treatment target. This allows the surgeon to easily insert the distal end of the imaging wire 2 into one of the cavities that is the treatment target.

### Modification 2-4

The distal end of the access tube 1 may be provided with a marker that is visible under X-ray observation. The surgeon can easily visually confirm the position of the distal end of the access tube 1 under X-ray observation.

### Modification 2-5

The endoscope may have a holding mechanism that fixes the access tube 1 to the forceps port 222 at the forceps port 222. If the access tube 1 is held by the holding mechanism, the position of the access tube 1 will be fixed even if the surgeon releases the access tube 1.

### Third Embodiment

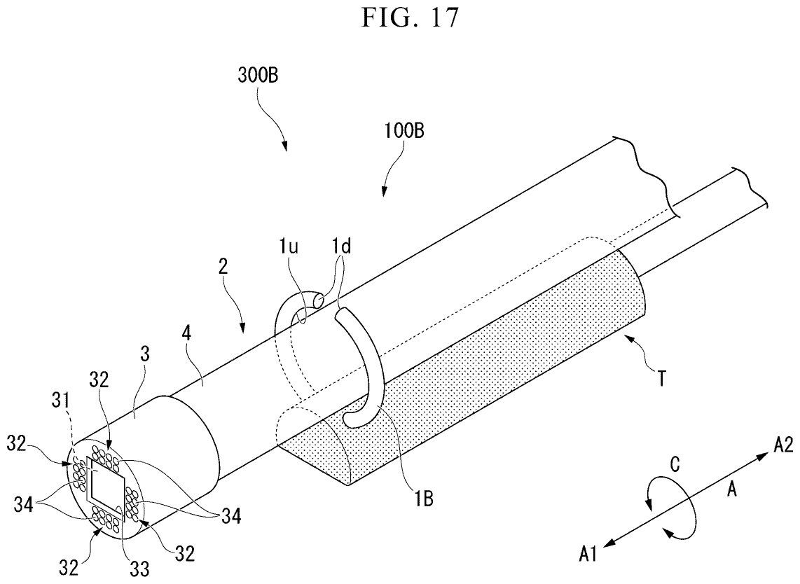

A bile duct and pancreatic duct treatment method according to a third embodiment of the present invention will be described with reference to FIGS. 17 to 19. In the following description, components that are common to those already described will be assigned the same reference numerals and duplicated descriptions will be omitted.

The bile duct and pancreatic duct treatment method according to this embodiment uses an endoscopic treatment system 300B. The endoscopic treatment system 300B includes an endoscope 200 and an endoscopic instrument 100B.

FIG. 17 is a diagram showing an endoscopic instrument 100B according to this embodiment.

The endoscopic instrument 100B (also referred to as instrument 100B) includes an access guide 1B and an imaging wire 2. In the following description, in the longitudinal direction A of the instrument 100B, the side that is inserted into the patient's body is referred to as the “distal side A1”, and the opposite side to the distal side A1 is referred to as the “base side A2”.

The access guide (access port) 1B is a C-shaped ring member that can be attached to the distal end of the treatment instrument T. The access guide 1B may be such that multiple access guides 1B are attached to the distal end of the treatment instrument T. The treatment instrument T to which the access guide 1B is attached can be inserted into the channel 230 of the endoscope 200. An internal space 1u through which the imaging wire 2 can be inserted is formed inside the access guide 1B. The inner diameter of the access guide 1B is, for example, 2 mm to 3 mm. Other treatment instruments such as a high-frequency knife and a cannula can also be inserted into the internal space 1u of the access guide 1B. The access guide 1B can guide the imaging wire 2 and other treatment tools that pass through the internal space 1u.

The access guide 1B has a cutout 1d in a part of the circumferential direction C. The surgeon can accommodate the imaging wire 2 in the internal space 1u by pushing the imaging wire 2 from the outside of the cutout 1d.

The access guide 1B is attached to the distal end of the treatment tool T. Therefore, the imaging unit 3 of the imaging wire 2 guided by the access guide 1B is disposed on the distal side A1 of the treatment tool T to which the access guide 1B is attached.

The access guide 1B may be attached slightly to the base side A2 from the distal end of the treatment tool T so that the imaging unit 3 can be disposed on the base side A2 of the distal end of the treatment tool T.

Next, a bile duct/pancreatic duct treatment method according to this embodiment will be described. Specifically, a procedure for observing and treating a diseased area D of the bile duct B will be described.

### <Step S3-1: Endoscope Insertion Step>

The surgeon inserts the distal end rigid section 211 of the endoscope 200 into the duodenum DU, as in step S1 of the first embodiment.

### <Step S3-2: Imaging Wire Insertion Step>

The surgeon inserts the imaging wire 2 into the common tube U, as in step S2 of the first embodiment.

As in the first embodiment, the imaging wire 2 has a smaller outer diameter than a conventional biliary-pancreatic endoscope, so the surgeon can insert the imaging wire 2 into the opening PA1 of the papilla PA without necessarily incising the opening PA1 of the papilla PA. Only in certain situations, such as when it is difficult to insert the imaging wire 2 into the opening PA1 of the papilla PA, can the surgeon incise the opening PA1 of the papilla PA using a cannula papilla incision knife or the like.

The surgeon inserts the distal end of the imaging wire 2 into the bile duct B of the treatment target, rather than the pancreatic duct P, by moving the distal end of the imaging wire 2 back and forth and rotating it while checking the image captured by the imaging unit 3. If the treatment target is a diseased area of the pancreatic duct P, the surgeon inserts the distal end of the imaging wire 2 into the pancreatic duct P of the treatment target rather than the bile duct B.

As in the first embodiment, the surgeon can easily distinguish between the bile duct B and the pancreatic duct P by inserting the imaging wire 2 into the common duct U while directly checking the image captured by the imaging unit 3 and insert the imaging wire 2 into one of the cavities that is the treatment target.

The surgeon may use a cannula to inject a contrast agent into the opening PA1 of the papilla PA before the imaging wire insertion step. The surgeon can check the distal end of the imaging wire 2 using the X-ray contrast image obtained by endoscopic retrograde cholangiopancreatography (ERCP) in addition to the image captured by the imaging unit 3.

### <Step S3-3: Observation Step>

The surgeon observes the diseased area D of the bile duct B while advancing and retracting the imaging wire 2 inserted into the bile duct B, as in step S3 of the first embodiment.

Similar to the first embodiment, the imaging wire 2 has a smaller outer diameter than a conventional biliary-pancreatic endoscope, so the surgeon can easily pass the imaging wire 2 through the stenosis. Therefore, the surgeon can easily observe the bile duct B using the imaging wire 2.

### <Step S3-4: Treatment Tool Insertion Step>

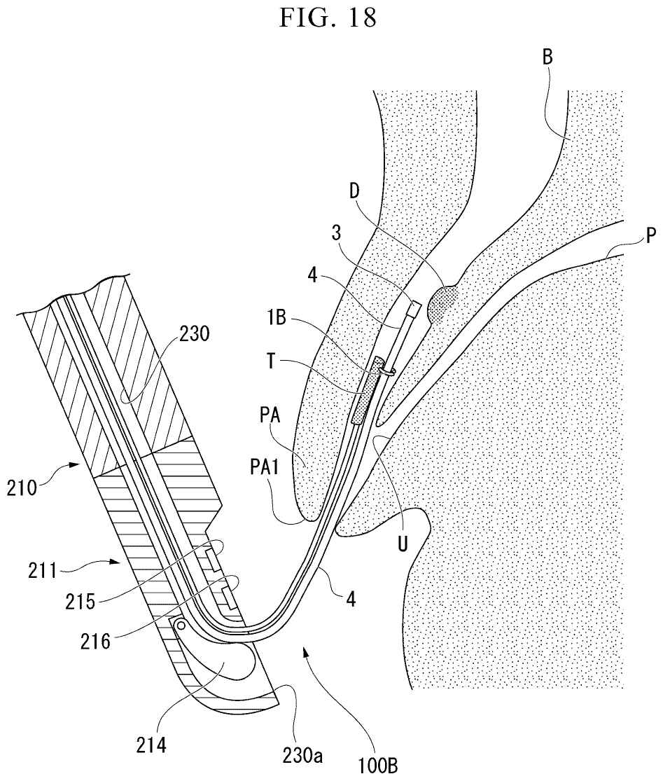

FIG. 18 is a diagram showing a treatment tool insertion step.

If the surgeon observes the diseased area D and determines that a biopsy diagnosis or treatment is necessary, the surgeon inserts a treatment tool T into the bile duct B along the imaging wire 2 that has already been inserted into the bile duct B. Specifically, the surgeon inserts the treatment tool T into the bile duct B by inserting the imaging wire 2 into the internal space 1u of the access guide 1B and moving the access guide 1B and the treatment tool T to the distal side A1 relative to the imaging wire 2.

The treatment tool T to which the access guide 1B is attached is positioned on the proximal side A2 of the imaging unit 3. Therefore, the treatment tool T is inserted into the luminal area observed by the imaging unit 3.

### <Step S3-5: Treatment Step>

The surgeon uses the treatment tool T to treat the diseased area D. As shown in FIG. 18, the surgeon treats the diseased area D with the treatment tool T positioned on the proximal side A2 of the imaging unit 3.

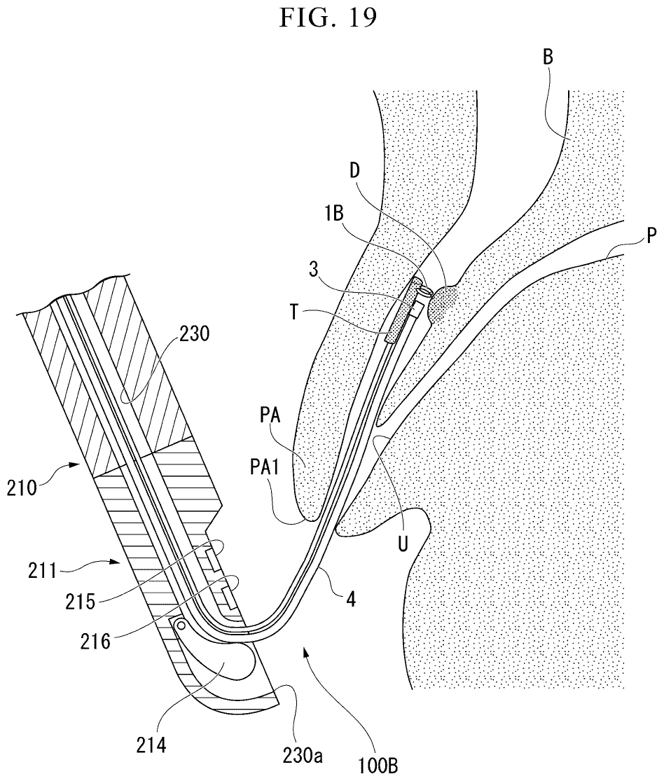

FIG. 19 is a diagram showing the treatment step.

As shown in FIG. 19, the surgeon may remove the treatment tool T from the imaging wire 2 and separate the imaging wire 2 and the treatment tool T. While checking the image captured by the imaging unit 3, the surgeon can treat the diseased area D using the treatment tool T separated from the imaging wire 2.

After completing the treatment, the surgeon removes the treatment tool T, the access tube 1, and the endoscope 200.

According to the bile duct/pancreatic duct treatment method and the endoscopic instrument 100B according to this embodiment, the endoscopic instrument 100B and the treatment tool T can be easily inserted into the bile duct B or the pancreatic duct. Since the treatment tool T to which the access guide 1B is attached and the imaging wire 2 are separate, only the imaging wire 2 can be inserted into the bile duct B first. The treatment tool T to which the access guide 1B is attached and the imaging wire 2 have smaller outer diameters than conventional bile duct/pancreatic endoscopes in which an imaging mechanism and a treatment tool port are integrally provided, so that the invasiveness in observation and treatment is low.

The third embodiment of the present invention has been described above in detail with reference to the drawings, but the specific configuration is not limited to this embodiment, and design changes and the like within the scope of the present invention are also included. Furthermore, the components shown in the above-described embodiment and modified examples can be appropriately combined to form a configuration.

What is claimed is:

1. A bile duct/pancreatic duct treatment method, comprising:
positioning an endoscope having a channel from a duodenum to an opening of a papilla;
inserting an imaging wire having an imaging unit at its distal end through the channel from the opening to a common duct;
inserting the imaging wire into one of cavities of a bile duct and a pancreatic duct;
observing the one of the cavities using the imaging unit; and
if treatment with a treatment tool is required, inserting an access port through which the treatment tool can be inserted along the imaging wire into the one of the cavities.

2. The bile duct/pancreatic duct treatment method according to claim 1, further comprising:
inserting the imaging wire into the one of the cavities based on an image captured by the imaging unit.

3. The bile duct/pancreatic duct treatment method according to claim 2, further comprising:
inserting the imaging wire into the one of the cavities based on an X-ray contrast image in addition to the image captured.

4. The bile duct/pancreatic duct treatment method according to claim 1, further comprising:
treating a diseased part of the one of the cavities with the treatment tool arranged on a distal side of the access port.

5. The bile duct/pancreatic duct treatment method according to claim 1, wherein
the access port is an access tube having a lumen through which the imaging unit can be inserted, and
the access tube is inserted into the one of the cavities by moving the access tube toward the distal side relative to the imaging wire that passes through the lumen of the access tube.

6. The bile duct/pancreatic duct treatment method according to claim 5, wherein
before inserting the imaging wire into the one of the cavities, the access tube is inserted up to the common duct.

7. The bile duct/pancreatic duct treatment method according to claim 6, further comprising:
inserting the imaging wire that passes through the access tube inserted up to the common duct into the one of the cavities.

8. The bile duct/pancreatic duct treatment method according to claim 7, further comprising:
inserting the access tube inserted up to the common duct into the one of the cavities by moving the access tube toward the distal end relative to the imaging wire.

9. The bile duct/pancreatic duct treatment method according to claim 1, wherein
the access port is attached to the treatment tool and is a ring-shaped access guide through which the imaging unit can be inserted, and
the access guide and the treatment tool are inserted into the one of the cavities by moving the access guide toward the distal end relative to the imaging wire through which the access guide is inserted.

10. The bile duct/pancreatic duct treatment method according to claim 9, further comprising:
treating the diseased part of the one of the cavities with the treatment tool arranged on a proximal side of the imaging unit.

11. The bile duct/pancreatic duct treatment method according to claim 9, further comprising:
separating the imaging wire inserted into one of the cavities from the treatment tool; and
treating the diseased part of the one of the cavities with the treatment tool.

12. An endoscopic instrument, comprising:
an imaging wire having an imaging unit at its distal end; and
an access port through which the imaging wire can be inserted,
wherein the imaging wire and the access port are relatively movable along a longitudinal direction of the endoscopic instrument.

13. The endoscopic instrument according to claim 12, wherein the access port is an access tube having a lumen through which the imaging wire can be inserted.

14. The endoscopic instrument according to claim 12, wherein the access port is attachable to a treatment tool and is a ring-shaped access guide through which the imaging wire can be inserted.

15. The endoscopic instrument according to claim 12, wherein
the imaging unit includes
an imaging section having a lens and an image sensor, and
an illumination section that irradiates light supplied from a light source device via an illumination fiber, and
the illumination section is disposed around the imaging section.
